(19) United States
(12) Patent Application Publication
(10) Pub. No.: US 2009/0216087 A1
(43) Pub. Date: Aug. 27, 2009

(54) Pivot Linkage Tightening Surgical Retractor Joint

(75) Inventor: Todd M. BJORK, River Falls, WI (US)

Correspondence Address: SHEWCHUK IP SERVICES, 3356 SHERMAN CT. STE. 102, EAGAN, MN, 55121, US

(73) Assignee: Minnesota Scientific, Inc., St. Paul, MN (US)

(21) Appl. No.: 12/037,820

(22) Filed: Feb. 26, 2008

(51) Int. Cl. A61B 1/32; A61B 17/08

(52) U.S. Cl. 600/201; 606/151

(57) ABSTRACT

A joint or clamp for a surgical retraction system uses a pivot linkage tightening mechanism. The pivot link primarily pivots to move one arm of the clamp relative to the other arm of the clamp. The handle contacts the clamp body at a friction interface of small radius to the handle pivot axis so little frictional torque is generated during tightening of the clamp. A handle pivot pin mostly translates above the handle pivot axis. The handle pivot pin has a center section which is offset, and the handle pivot pin can be received in any of numerous circumferential positions to absorb the tolerance stack for the clamp, resulting in a consistent and precise tightening mechanism achieved by a small throw of a short handle.

### CROSS-REFERENCE TO RELATED APPLICATION(S)

None.

### BACKGROUND OF THE INVENTION

The present invention relates to the field of surgical tools, and particularly to the design and manufacture of surgical retractor systems. Surgical retractor systems have long been used during surgery to bias and hold tissue in a desired position. In many retractor systems, clamps are used which have a loosened position in which the post, shaft, retractor blade and/or other portions of the assembly can be easily moved and a tightened position in which the clamped connection is held rigid.

Numerous such surgical retractor clamps exist in the prior art. Some surgical retractor clamps are actuated by a rotational, screw-type tightening action, while others are tightened by a throw of a handle. For all these clamps, the basic clamping force is generated because the handle movement shortens or lengthens the distance between two portions of the clamp. Often the compressive or tensile force of clamp tightening is supported by a clamp bolt extending through two arms of the clamp. In one type of clamp having a handle throw, the clamp bolt is tensioned or compressed by a cam or eccentric section on the end of a handle which acts against either a bearing surface or an opening in the clamp bolt. During the throw of the handle, the effective radius of the cam from the handle pivot point to the bearing surface changes, or the height of the eccentric relative to the handle pivot point changes, pulling or pushing on the clamp bolt. Examples of surgical retractor clamps having a pivot handle which operates a cam or eccentric portion include those of U.S. Pat. Nos. 5,727,899, 5,741,210, 5,792,046, 5,888,197, 5,897,087, 5,899,627, 6,017,008, 6,033,363, 6,042,541, 6,264,396, 6,645,141, 6,663,563 6,790,177, and U.S. Patent Publication nos. 2005/0080321.

The force required to tighten a surgical retractor clamp should be sufficiently small that a surgeon can tighten or loosen the clamp easily by hand, preferably with a single hand. One way to lessen the hand force required to tighten the clamp is to lengthen the handle, so the handle throw provides a greater mechanical advantage. However, particularly when used near the surgical arena, surgical retractor clamps should have a low profile so as to minimize intrusion into the line of sight or and maximize access to the surgical site. While longer handles may lessen the hand force required to switch between loosened and tightened positions, the longer handle may impede on the surgical site. At the same time, the clamp should support a sufficient load when tightened that no sliding or repositioning of the connected components will occur even under a large retraction force load and even if bumped or jostled during the surgery.

Surgical retractor systems must be robust and strong, as even a possibility of failure during use is not tolerated. Surgical retractor assemblies should be readily reusable, including sterilizable, for use in multiple surgeries. Surgical retractor systems should maintain a relatively low cost. Improvements in surgical retractor systems can be made in keeping with these goals.

### BRIEF SUMMARY OF THE INVENTION

The present invention is a clamp or joint for a surgical retraction system which uses a pivot linkage tightening mechanism. The pivot link primarily pivots to move one arm of the clamp or joint relative to another arm. Through utilizing the pivot link, the frictional torque generated between the handle and the clamp body can be reduced relative to prior art cammed clamps. In particular, a frictional moment arm from the frictional contact to the handle pivot axis is shorter than a clamping moment arm from the pivot link coupling point to the handle pivot axis. In a separate aspect, the tolerance stack for the clamp is absorbed by an offset in a handle pivot pin. In another separate aspect, the clamp includes a lip capture mechanism for slidable attachment to a rectangular cross-sectioned bar of a retractor frame, such as to a Bookwalter-type frame.

### BRIEF DESCRIPTION OF THE DRAWINGS

FIG. 1 is a perspective view of a surgical retractor clamp with a pivot linkage tightening mechanism in accordance with the present invention, shown in a tightened position, with the retractor shaft receptacle canted forward and to the left.

FIG. 2 is a side elevational view of the surgical retractor clamp of FIG. 1, shown in a loosened position, with the retractor shaft receptacle uncanted.

FIG. 3 is a mid-plane cross-sectional side view of the surgical retractor clamp of FIGS. 1 and 2 in the loosened position, with the retractor shaft receptacle uncanted.

FIG. 4 is a cross-sectional side view of the surgical retractor clamp of FIGS. 1-3 in the tightened position, taken along the same mid-plane as FIG. 3, along lines 4-4 in FIG. 1 except showing the retractor shaft receptacle uncanted.

FIG. 5 is a cross-sectional side view of the pivot linkage tightening portion of the clamp in the loosened position, superimposing the pivot linkage in dotted lines.

FIG. 6 is a geometrical drawing for analyzing the tightening profile of the clamp of FIG. 5 in the loosened position.

FIG. 7 is a cross-sectional side view of the pivot linkage tightening portion of the clamp in the tightened position, taken along lines 7-7 in FIG. 1, superimposing the pivot linkage in dashed lines, and showing the loosened position in dotted lines.

FIG. 8 is a geometrical drawing for analyzing the tightening profile of the clamp of FIG. 5 in the tightened position.

FIG. 9 is a cross-sectional side view of a first alternatively shaped ear recess for the clamp.

FIG. 10 is a cross-sectional side view of a second alternatively shaped ear recess for the clamp.

FIG. 11 is a perspective view of the preferred handle pivot pin.

FIG. 12 is a side view of the preferred handle pivot pin of FIG. 11.

FIG. 13 is an end view of the preferred handle pivot pin of FIGS. 11 and 12.

FIG. 14 is a cross-sectional view of a first preferred clamp of FIGS. 1-4 taken along lines 14-14 in FIG. 2, and of a second preferred clamp, on a Bookwalter/Codman type ring.

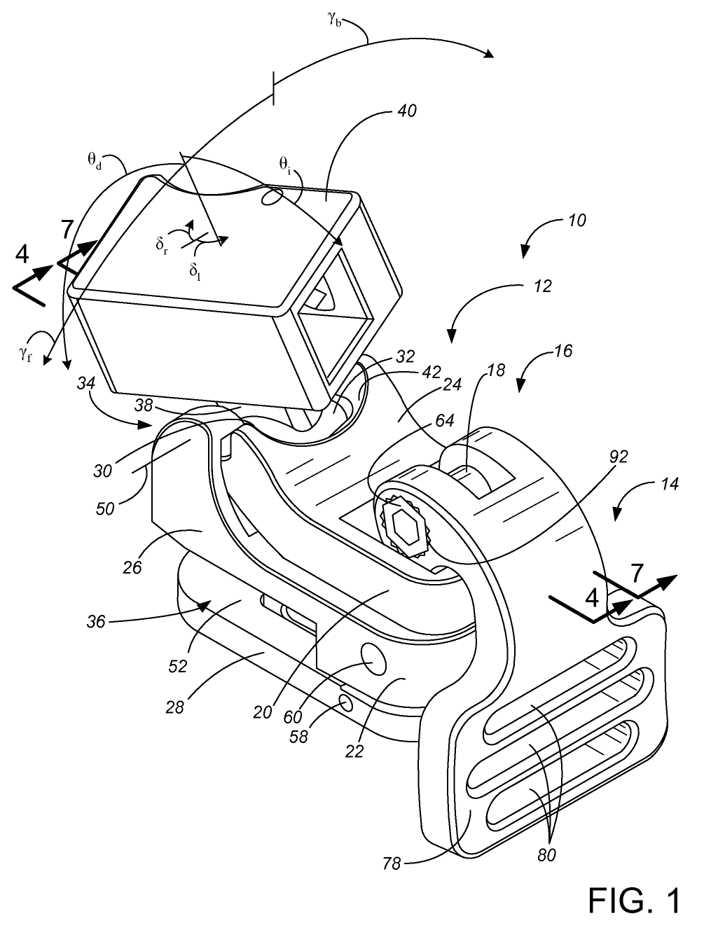

While the above-identified drawing figures set forth preferred embodiments, other embodiments of the present invention are also contemplated, some of which are noted in the discussion. In all cases, this disclosure presents the illustrated embodiments of the present invention by way of representation and not limitation. Numerous other minor modifications and embodiments can be devised by those skilled in the art which fall within the scope and spirit of the principles of this invention.

### DETAILED DESCRIPTION

A surgical retractor joint or clamp 10 representing a preferred embodiment of the present invention includes a clamp or joint body 12 and an actuating handle 14. The handle 14 operates on a pivot linkage tightening mechanism 16 having a pivot link 18 which, in this embodiment, is pulled into tension during tightening of the clamp 10. The body 12 includes two arms 20, 22 which are pulled toward each other by the tension in the pivot link 18 to tighten the clamp 10. The pivot link 18 extends through openings in the middle of each of the arms 20, 22. Other embodiments might place the pivot link 18 in compression in the tightened position. For example, the pivot linkage tightening mechanism 16 of the present invention could alternatively be used with any of the clamp bodies of U.S. Pat. Nos. 4,718,151, 4,949,707, 5,020,195, 5,242,240, 5,727,899, 5,741,210, 5,792,046, 5,888,197, 5,897,087, 5,899,627, 6,017,008, 6,033,363, 6,042,541, 6,264,396, 6,645,141, 6,663,563, 6,736,755 and 6,790,177, and U.S. Patent Publication Nos. 2005/0059866, 2005/0080321 and 2005/0272981 all incorporated by reference. Many other clamp body designs could also incorporate the pivot linkage tightening mechanism 16 of the present invention.

The clamp body 12 includes three primary components: a top clamp 24, a central stop member 26, and a lower jaw 28. Throughout this specification, the terms “top”, “lower” and similar directional terms are applied based upon the orientation of the clamp 10 shown in the figures; though most commonly used in this orientation, the clamp 10 can be used in any orientation, including being flipped over so the top clamp 24 is lower in elevation than the lower jaw 28.

With the preferred clamp body 12, an upper clamping location 30 changes dimensions to tighten about a ball 32 in a ball-in-socket joint 34. A lower clamping location 36 attaches about notched rectangular bar stock (shown in FIG. 14). While the preferred clamp 10 does not use the tightening force of the pivot linkage tightening mechanism 16 to tighten on such rectangular bar stock, modifications could easily be made such that the force of the pivot linkage tightening mechanism 16 operates on the lower clamping location 36 as well as the upper clamping location. Other clamp body styles may clamp about rods other than a ball or bar stock while fully utilizing the pivot linkage tightening mechanism 16 of the present invention.

The ball 32 is generally spherical, with a ball shaft 38 extending off one side of the ball 32. The ball shaft 38 extends to a rectangular retractor shaft receptacle 40 such as known in the art for holding and applying tension on a square, notched Bookwalter/Codman type retractor shaft (not shown). Examples of such square, notched Bookwalter/Codman type retractor shafts are shown in the various patents of John R. Bookwalter et al., such as U.S. Pat. Nos. 4,254,763, 4,421,108, 4,424,724, 4,467,791, 5,375,481, 5,520,608, 6,241,659, 6,530,882 and 6,808,493, all incorporated by reference, as originally made and marketed by Codman & Shurtleff, Inc. of Randolph, Mass. Additional examples include those shown in U.S. Pat. Nos. 1,919,120, 1,963,173, 4,434,791, and 5,520,610, all incorporated by reference. In the preferred embodiment, the ball shaft 38 is rectangular and received in a ball shaft opening 42 jointly defined by the top clamp 24 and the central stop member 26. The relative size of the ball shaft 38 as compared to the ball shaft opening 42 permits the ball 32, ball shaft 38 and receptacle 40 to pivot downward through a maximum declining pitch angle θ_{d} before the ball shaft 38 contacts the central stop member 26, and permits the ball 32, ball shaft 38 and receptacle 40 to pivot upward through a maximum inclining pitch angle θᵢ before the ball shaft 38 contacts the top clamp 24. The relative size of the ball shaft 38 as compared to the ball shaft opening 42 also permits the ball 32, ball shaft 38 and receptacle 40 to twist about the ball shaft axis 44 through a left twist (yaw) angle δ₁ and through a right twist (yaw) angle δᵣ. The relative size of the ball shaft 38 as compared to the ball shaft opening 42 also permits the ball 32, ball shaft 38 and receptacle 40 to pivot forward and backward through maximum roll angles γ_{f} and γ_{b}. In the preferred embodiment, the maximum declining angle θ_{d} is about 65°, the maximum inclining angle θᵢ is about 30°, the maximum left and right twist angles δ₁ and δᵣ are both about 50°, and the maximum roll angles γ_{f} and γ_{b} are both about 25°. Modifications to the relative shapes and sizes of the ball shaft 38 and ball shaft opening 42 can permit wide variations to these angles, as desired for the degree of joint flexibility needed for the clamp 10.

The preferred socket of the ball-in-socket joint 34 is provided on the bottom side by an upwardly open concave recess 46 on the central stop member 26 and on the top side by a downwardly open concave recess 48 on the top clamp 24. The top clamp 24 preferably pivots relative to the central stop 26 about a hinge axis 50. The top clamp 24 thus provides a top arm 20 and the central stop 26 provides a bottom arm 22 around the ball 32, and pulling the top arm 20 toward the bottom arm 22 tightens the clamp body 12 about the ball 32.

The preferred opening 52 of the lower clamp 36 is provided on the top side by the central stop 26 and on the bottom side by the lower jaw 28. The central stop 26 may include a half-moon extension 54 sized to mate into the arc notches 53 of Bookwalter/Codman type ring stock 55 (shown in FIG. 14). Alternatively, as shown in the second embodiment depicted in FIG. 14, the half-moon extension 54 may be omitted with the central stop 26 having a flat face 57 which contacts the arc notches 53 of Bookwalter/Codman type ring stock 55. On the opposite side of the opening 52, the lower jaw 28 includes a lipped end 56 to clip around the Bookwalter/Codman type ring stock 55.

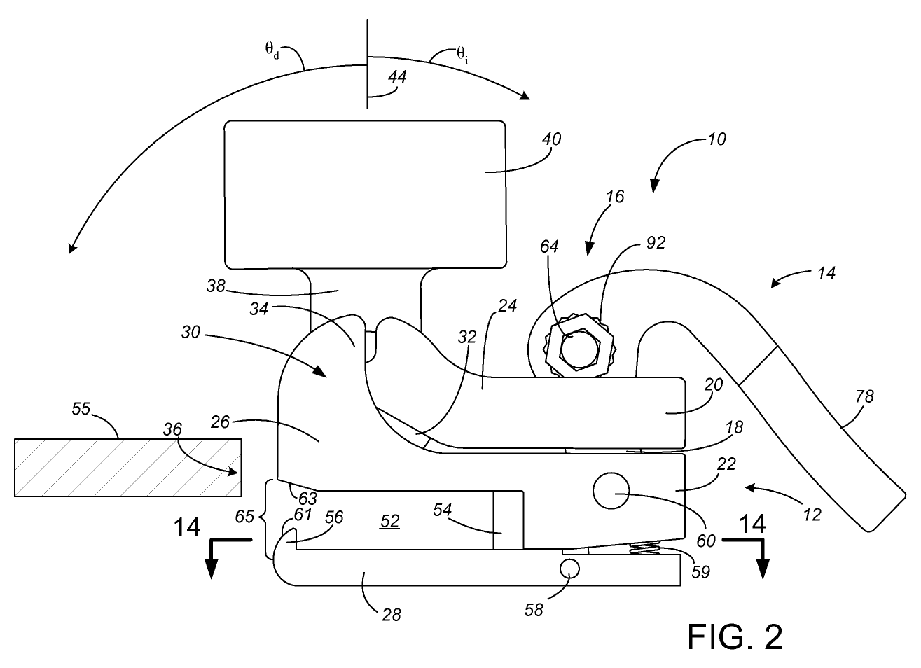

The lower jaw 28 preferably pivots relative to the central stop 26 about a jaw connection pin 58. A compression spring 59 is housed between the lower jaw 28 and the central stop 26. In the preferred embodiment, the compression spring 59 can be compressed to deflect the lipped end 56 beneath the bottom surface of the Bookwalter/Codman type ring stock 55 with only a few pounds of force. As best shown in FIG. 2, the lipped end 56 has a sloped entry surface 61. When the surgeon desires to place the clamp 10 on the Bookwalter/Codman ring 55, the surgeon may merely push the clamp 10 onto the desired location. With the light force provided by the compression spring 59, when the sloped entry surface 61 contacts the ring 55, the ring 55 easily pushes the sloped entry surface 61 downward and progresses past the lipped end 56 into the opening 52. When the clamp 10 is fully advanced onto the ring 55, the compression spring 59 snaps the lipped end 56 upward, wrapping around the ring 55 and retaining the clamp 10 on the ring 55.

In addition to the sloped entry surface 61 of the lipped end 56, the clamp body 12 preferably also has a sloped entry surface 63. In placing the clamp 10 on the Bookwalter/Codman ring 55, if the ring 55 contacts the sloped entry surface 63 of the clamp body 12, the sloped entry surface 61 will push the clamp 10 upward, with the ring 55 sliding downward along the sloped entry surface 63 to deflect the lower jaw 28 out of its way. Once the ring 55 is fully seated in the opening 52, the lower jaw 28 clicks up into place with the lipped end 56 contacting the fourth side of the ring 55. With both the sloped entry surface 63 of the clamp body 12 and the sloped entry surface 61 of the lipped end 56, the surgeon has a large target area 65 for hitting the ring 55 to have the clamp 10 simply and easily snap onto the ring 55.

In the embodiment with the flat face 55, the clamp 10 can then be slid along the ring 55 as shown by arrows 67. In the embodiment with the half-moon extension 54, the half-moon extension 54 prevents such sliding on the ring 55. To reposition the clamp 10 on the ring 55, the surgeon need only depress the finger button portion 69 of the lower jaw 28 to pivot the lipped end 56 beneath the ring 55, and then the half-moon extension 54 can be disengaged from its notch 53, permitting repositioning of the clamp 10. With the light compression spring 59, only a pound or two of force on the finger button portion 69 is sufficient to disengage the clamp 10 from the ring 55. Even without the half-moon extension 54, when the retractor system is fully assembled and in use, the force of retraction pulls the clamp 10 more tightly onto the ring 55, so the clamp 10 frictionally engages the ring 55 and slides only minimally relative to the ring 55 during use.

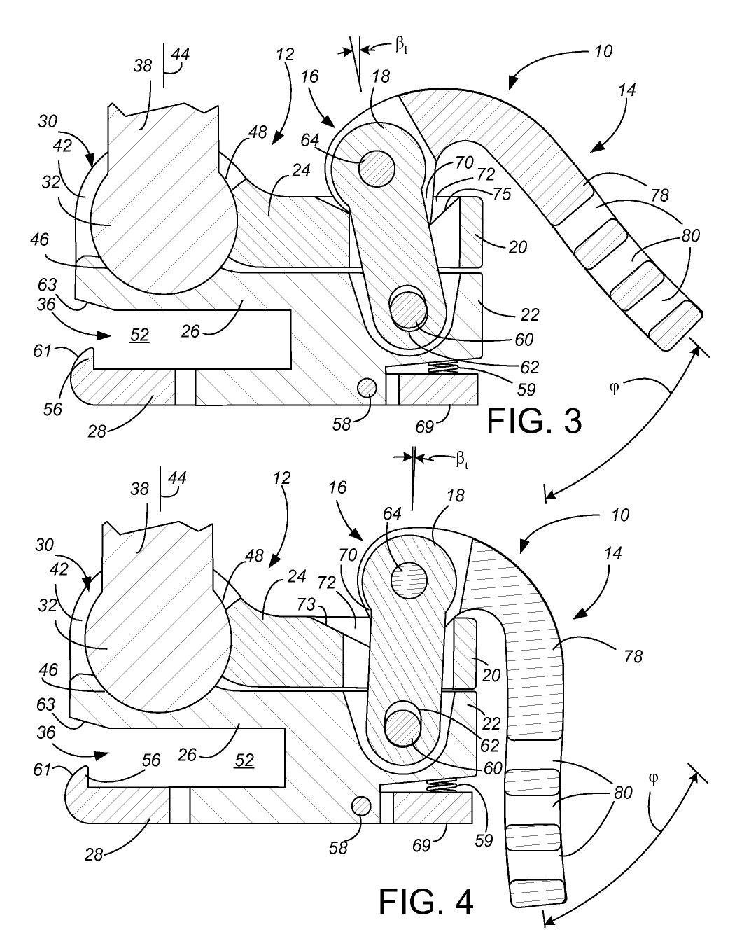

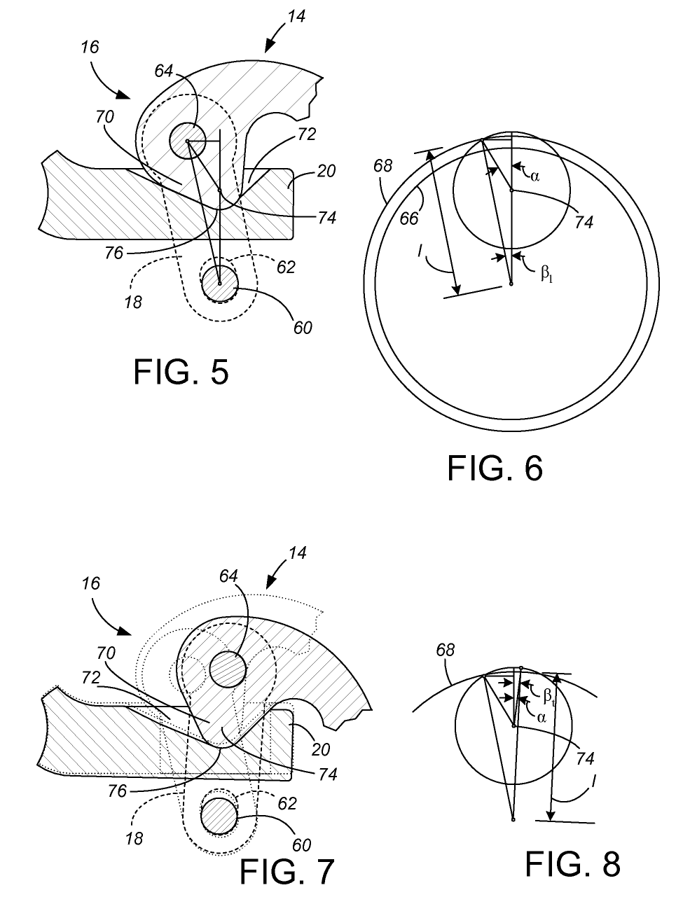

The tightening mechanism 16 of the present invention includes a pivot link 18, and the operation of the pivot linkage tightening mechanism 16 is best shown in FIGS. 3-8. The bottom end of the pivot link 18 is pivotally connected to the central stop 26 by a stop pivot pin 60 extending through an oblong pin opening 62 in the pivot link 18. In the preferred embodiment, the stop pivot pin 60 is about 0.065 inches in radius. The top end of the pivot link 18 is pivotally connected to the handle 14 by a handle pivot pin 64. In the preferred embodiment, the handle pivot pin 64 is about 0.094 inches in radius. Showing the amount of play in effective pivot link length provided by the oblong pin opening 62, the handle pivot pin 64 is constrained to pivot between two pivot arcs 66, 68 shown in FIG. 6. In the preferred embodiment, the oblong pin opening 62 defines the effective pivot link length at a maximum of about 0.507 in. and a minimum of about 0.480 in.

The proximal end of the handle 14 includes two ears 70 that mate into appropriately shaped ear recesses 72 of the top clamp 24. In the preferred embodiment, the mating shapes between the ear recesses 72 and the ears 70 cause the handle 14 to pivot about a handle pivot axis 74 when the handle 14 is thrown to tighten the clamp 10. The exact location of the handle pivot axis 74 is defined by the radius of the end of the handle ears 70 as interacting with the radius of the bottom of the ear recesses 72. In the preferred embodiment, the handle ear radius is only about 0.077 in. The ear recesses 72 are formed with a matching radius of about 0.077 in. With these matching radii, the handle pivot axis 74 remains fixed relative to the top clamp 24 during tightening of the clamp 10.

The location of the handle pivot axis 74 relative to the center of the handle pivot pin 64 determines the amount of mechanical advantage being provided by the length of the handle 14. In the preferred embodiment, the handle pivot axis 74 is spaced about 0.185 inches away from the center of handle pivot pin 64. During the pivot throw, the angle a of this handle ear line changes from about 33° to about −7° relative to vertical. With these angles α, the handle pivot pin 64 primarily translates relative to the clamp body 12 during tightening of the clamp 10.

The ear recesses 72 define a loosened stop 73 and a tightened stop 75 for the handle 14. The stops 73 and 75 mate with corresponding sides of the handle ears 70 to prevent over-rotation of the handle 14 in a loosening direction and to prevent over-rotation of the handle 14 in a tightened direction, thereby defining the handle throw angle φ. In the preferred embodiment, the handle throw angle φ is only about 40°. During the tightening throw, the pivot link 18 moves from a loosened angle β₁ taken relative to vertical over the stop pivot pin 60 to a tightened angle βₜ taken relative to vertical over the stop pivot pin 60. In the preferred embodiment, the loosened angle β₁ is about 11.5° and the tightened angle βₜ is about −2.5°. During tightening of the clamp 10, the pivot link 18 moves slightly axially relative to upper arm 20, but the handle receiving end of the pivot link 18 around the handle pivot pin 64 moves much more laterally, i.e., transverse to the axis of the pivot link 18. Due to this lateral movement of the handle pivot pin 64 and the pivoting of the pivot link 18, the vertical component of the effective length of the pivot link 18 changes during the handle throw according to sin β. That is, in the preferred embodiment the effective vertical length component has a maximum length of 0.507*sinβ. Depending upon tolerances and position of the ear recesses 72 relative to the central stop 26 and stop pivot pin 60 when the clamp 10 is in the loosened position, a part of the throw may be used simply to run out the slight amount of play provided by the oblong pin opening 62. With the pivot link 18 primarily pivoting rather than moving vertically during tightening, the pivot link coupling point defined by the handle pivot pin 64 remains vertically oriented on the same side of (i.e, at a higher elevation than) the handle pivot axis 74 during the entire handle throw.

In contrast to the cammed/eccentric handles and clamp bolts of prior art designs which operate to raise the clamp bolt primarily vertically, the tightening mechanism 16 of the present invention includes a pivot link 18 and the tightening profile involves the change in angle of the pivot link 18. When the various forces acting against the tightening throw of the handle 14 are considered, the spring action of the clamp body 12 is primary. Depending upon the design of the clamp body 12, the spring force increases in a generally proportional manner based upon the amount of deflection of the arms 20, 22 of the clamp 10. In contrast to many prior art clamps, the amount of deflection of the arms 20, 22 of the clamp 10 is not a linear function of the angle of the handle 14 during its throw, nor even a simple trigonometric function of the angle of the handle 14 during its throw. Instead, the mechanical advantage obtained by the pivot link 18 of the present invention involves both the angle of the handle 14 (specifically the angle of the line between the axis 74 of handle rotation and the center of handle pin 64) and the angle of the pivot link 18. The rate of arm deflection change per change in handle position varies based upon where the handle 14 is in its throw, in a way that can be adjusted differently and more effectively than a simple circular cam or eccentric. Specifically, the preferred clamp 10 gives a greater amount of tightening action while the torque spring force is low, and then a significantly greater and increasing mechanical advantage for tightening when the torque spring force becomes greater. This changing mechanical advantage can be seen by comparing the geometrically calculated spring force and spring force torque versus handle throw angle in equal increments during the throw of the preferred embodiment, as follows:

| | Percent | Pivot | Elevation of Handle | Elevation of | Spring | Torque of |
|---|---|---|---|---|---|---|
| Handle | of total | linkage | Pivot Pin relative to | Handle Pivot | force | Spring |
| angle to | throw | angle | Stop Pivot Pin | Axis relative to | (% of | Force (% of |
| vertical | (φ %) | (β) | (1 * sinβ) | Stop Pivot Axis | maximum) | Maximum) |
| 40 | 0 | 11.5 | 0.4971 | 0.3424 | 0 | 0 |
| 36 | 10 | 10.2 | 0.4992 | 0.3379 | 23 | 53 |
| 32 | 20 | 8.8 | 0.5012 | 0.3340 | 43 | 86 |
| 28 | 30 | 7.4 | 0.5029 | 0.3306 | 60 | 100 |
| 24 | 40 | 6.1 | 0.5043 | 0.3279 | 74 | 99 |
| 20 | 50 | 4.7 | 0.5054 | 0.3257 | 85 | 87 |
| 16 | 60 | 3.3 | 0.5064 | 0.3242 | 93 | 66 |
| 12 | 70 | 1.8 | 0.5069 | 0.3231 | 98 | 38 |
| 8 | 80 | 0.4 | 0.5072 | 0.3227 | 100 | 8 |
| 4 | 90 | −1.1 | 0.5071 | 0.3229 | 99 | −2 |
| 0 | 100 | −2.5 | 0.5067 | 0.3236 | 96 | −5 |

This calculation of spring force torque assumes no play in the length of the pivot link 18 (such as provided by the oblong hole 62), and similarly assumes no play or tolerance adjustments between the various components. This calculation of spring force torque similarly assumes that the clamp body 12 acts as an ideal spring of constant spring coefficient. While these assumptions don't hold entirely true in real life situations as far as the exact values provided, the macro trends and proportions exemplified by these calculated values do apply in real life situations.

A macro review of the torque of the calculated spring force of the clamp body 12 as a function of handle position reveals that the handle torque required to tighten the clamp 10 increases steeply from the loosened position, reaching over half of the maximum torque in a mere 4° of handle throw. From there, the tightening torque plateaus nicely over the next about 20° of handle throw. As the clamp 10 reaches its tightened position (where frictional forces discussed below are greatest), the spring torque required for further tightening decreases. At the end of the tightening throw, the spring torque actually serves to advance the handle throw, so the spring torque itself helps to maintain the clamp in a tightened position.

The above tabulation of spring force and spring force torque does not yet consider the effects of friction on the clamp 10. As important as the benefits to the profile of the torque vs. handle throw angle curve, the pivot linkage tightening mechanism 16 of the present invention also minimizes the friction which must be overcome during the tightening throw. Prior art clamp designs which utilize cams in the tightening mechanism have generally not properly considered and compensated for the effects of friction.

In the preferred embodiment, there are three frictional interfaces: between the stop pivot pin 60 and the pivot link 18, between the handle pivot pin 64 and the pivot link 18, and between the handle ears 70 and the ear recesses 72. During the handle throw, friction in each of these three locations increases as a nearly linear function of spring force between the arms 20, 22 of the clamp body 12. Each of these frictional forces acts through a moment arm to determine the torque required of the handle throw to overcome friction. More particularly, the frictional interfaces of the pins 60, 64 produce a frictional torque tied to tension forces bourne by the pivot link 18, multiplied by the diameter of each pin 60, 64. The frictional interface between the handle 14 and the top clamp 24 produces a frictional torque tied to the spring force of the clamp body 12, multiplied by the distance from the pivot axis 74 of the handle 14 to the contact interface 76 between the ears 70 and the ear recesses 72. The frictional moment arm from the pivot axis 74 to the contact interface 76 is shorter than the clamping moment arm from the handle pivot axis 74 to the axis of the handle pivot pin 64. Largely because the distance from the pivot axis 74 of the handle 14 to the contact interface 76 between the ears 70 and the ear recesses 72 is much less than the typical cam radius of prior art cam actuated clamps, the present invention minimizes the frictional torques witnessed during tightening of the clamp 10, making it easier to tighten the clamp 10 with a small angle throw (p of a short, compact handle 14. The pins 60, 64 can have as small a diameter as possible while still being adequately strong to bear the tension load of the pivot link 18.

It will be seen then that the pivot linkage tightening mechanism 16 of the present invention provides an elegant solution to maximizing clamp tightening with the smallest handle 14 and shortest handle throw (p possible. Both the mechanical advantage provided and the friction torque to be overcome proceed through curves such that the preferred embodiment achieves a very tightly binding clamp 10 with acceptably low thumb force on the handle 14. The handle torque is still sufficiently great that a widened thumb area 78 is provided on the handle 14 for application of this torque. This widened thumb area 78 may include ornamental openings 80 such as taught by U.S. patent application Ser. No. 29/280,993, incorporated by reference.

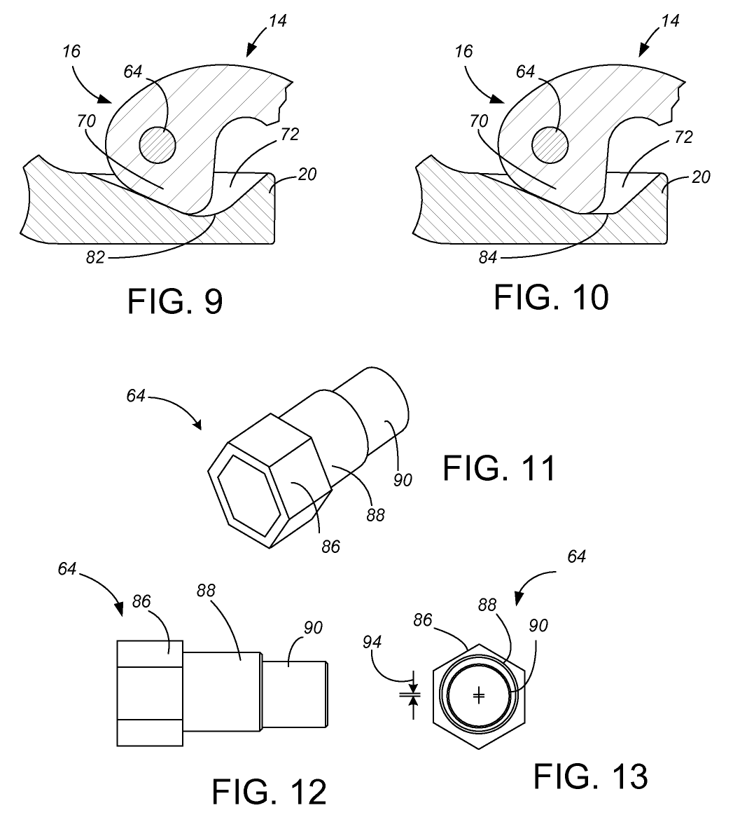

If desired, the frictional torque may be reduced even further by providing a rolling contact between the handle ears 70 and the ear recesses 72. For instance, if the ear recesses 72 have a more widely curved middle section 82 such as the recess curve shown in FIG. 9, or if the ear recesses 72 have a flat middle section 84 such as shown in FIG. 10, then the handle 14 tends to roll during tightening rather than slide against the handle ears 70. With a rolling contact, the handle pivot axis 74 will translate relative to the top clamp 24 during tightening of the clamp 10.

Still further reductions in frictional torque could be obtained by providing a rolling contact between the pins 60, 64 and their respective mating surfaces. Whether such rolling contacts can be effective depends greatly on the length 1 and angles α, β of the pivot link 18 and handle ear line and the respective mating frictional surfaces. Each rolling contact has a tendency to add a level of “slop” to the completed, assembled clamp 10, particular dangerous in the loosened position. That is, when the pivot link 18 supports no tightening force, rolling contacts have the possibility of rattling around, possibly becoming placed in a slightly offset location relative to the desired start position for the rolling contact, which can then result in frictional binding during tightening of the clamp 10. In the preferred embodiment, the advantage obtained by reducing the radius of the handle ears 70 to be equal to the radius of the pins 60, 64 was found sufficient to reduce the throw friction to an acceptable level, without making a rolling contact between the handle ears 70 and the ear recesses 72.

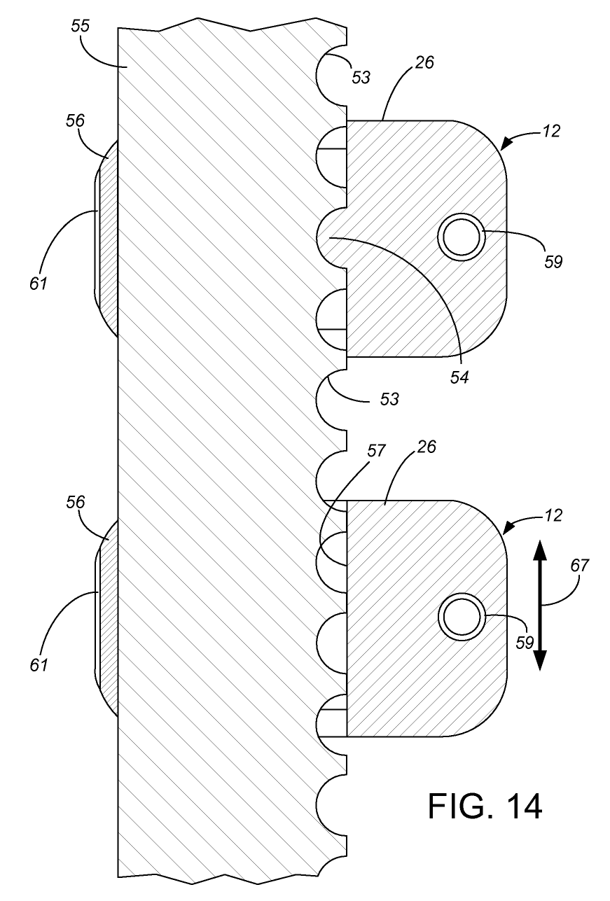

As mentioned earlier, the calculations of spring force torque and handle throw given above do not take the effects of tolerances into consideration. In constructing the clamp 10, the critical factor affecting spring force involves the changing elevation of the handle pivot axis 74 relative to the stop pivot pin 60. Numerous tolerances affect this value, including tolerances on the diameter of the stop pivot pin 60 and the handle pivot pin 64, on the oblong pin opening 62, on the opening for the stop pivot pin 60 in the central stop member 26, on the openings for the handle pivot pin 64 in the top clamp 24 and the pivot link 18, on the location of the upwardly open concave recess 46 in the central stop member 26, on the location of the downwardly open concave recess 48 in the top clamp 24, on the diameter of the ball 32, on the radius of the handle ears 70, and on the location of the ear recesses 72 in the top clamp 24. Given that the total change in elevation of the handle pivot axis 74 relative to the stop pivot pin 60 during the tightening throw of the handle 14 is only about 0.020 inches, very precise machining is required unless a mechanism is added to the clamp 10 to adjust for the tolerance stack. FIGS. 11-13 show another separate aspect of the invention, the preferred design of the handle pivot pin 64 to adjust for the tolerance stack.

The handle pivot pin 64 includes a flatted head 86, a cylindrical central section 88, and a cylindrical end 90. The head 86 mates into a flatted opening 92 in the top clamp 24. The flatted opening 92 in the top clamp 24 has multiple circumferential positions in which it can receive the head 86. The end 90, which is axially aligned with the flatted head 86, is press fit into a cylindrical opening (not shown) on the other side of the top clamp 24. The central section 88 is axially offset by an offset 94 relative to the axis of the head 86 and the end 90. In the preferred embodiment, the head 86 is hexagonal, while the flatted opening 92 is an 18-pointed star shape for receiving the hexagonal head 86 in any of 18 positions, each position spaced 20° from the next. The star shape of the flatted opening 92 is preferably rotationally offset (such as by 10°), so each of the 18 positions results in a different amount that the offset 94 changes the effective length of the pivot link 14. The circumferential position of the hexagonal head 86 when placed into the flatted opening 92 thus determines one of 18 different values for the effective length and exact angle β of the pivot link 14. During assembly of the clamp 10, the various component parts are positioned, and then the last step is selecting the circumferential position of the handle pivot pin 64 to best accommodate for the tolerance stack so the desired tension is best placed on the clamp in the loosened and tightened positions. The preferred embodiment uses a first value for the offset 94 of 0.005 inches, which can adjust for a maximum of nearly 0.010 inches variation in the collective tolerances.

If none of the 18 different rotational positions of the 0.005 offset handle pivot pin 64 makes the perfect fit desired, handle pivot pins with other amounts of offset can be used. Preferred amounts of offsets 94 are in the range of 0.001 to 0.020 inches. For instance, handle pivot pins 64 having precisely machined offsets 94 (say, for example 0.0060, 0.0070 and 0.0080 inches), used at a circumferential position offset of 95° relative to the pivot link 14, permit adjustments of 0.00052, 0.00053 and 0.00054 to the effective length of the pivot link 14. Depending upon how tight manufacturing tolerances vary from part to part within a production run, selection of the appropriate length of offset 94 coupled with the proper circumferential positioning of the offset 94 allows essentially infinitesimal adjustment of the clamp tightening mechanism even given significant tolerance differences between the designed and produced dimensions. If desired, each clamp 10 may be assembled around the ball 32 except for placement of the handle pivot pin 64, and the exact desired positioning of the handle pivot pin relative to the pivot link 14 may be optically scanned to computer measure and determine the ideal offset 94 and circumferential positioning of the handle pivot pin 64 as each clamp 10 proceeds in an assembly line.

The pivot linkage tightening mechanism 16 provided by the present invention can be achieved at low cost and with few parts. Each of the components can be formed of surgical stainless steel or similar materials as known in surgical retractor art. The clamp 10 with the inventive pivot linkage tightening mechanism 16 is accordingly easy to manufacture, is easy to sterilize, is strong and robust, and provides a low surgical profile in all modes of use. The force required to tighten the clamp 10 is minimized so as to make it possible to fully tighten the clamp 10 with a short, thumb operable handle 14 through a small tightening throw.

Although the present invention has been described with reference to preferred embodiments, workers skilled in the art will recognize that changes may be made in form and detail without departing from the spirit and scope of the invention.

1. A tightenable joint for a surgical retraction system, the joint comprising:
a clamp body defining a first opening for receiving a clamped rod, the clamp body comprising a first arm and a second arm operatively coupled together to change dimensions of the first opening to receive the clamped rod in a loosened clamp position or in a tightened clamp position;
a pivot link supporting a tightening force between the first arm and the second arm when the clamp is in the tightened clamp position;
a rigid handle pivoting relative to the clamp body about a handle pivot axis between the loosened clamp position and the tightened clamp position, the rigid handle being coupled to the pivot link at a pivot link coupling point, the rigid handle making frictional contact relative to the clamp body at a frictional bearing point, with a frictional moment arm from the frictional contact to the handle pivot axis and a clamping moment arm from the pivot link coupling point to the handle pivot axis, wherein the frictional moment arm is shorter than the clamping moment arm.

2. The tightenable joint of claim 1, wherein the first arm and the second arm each have a pivot link hole therein, wherein the pivot link extends through the pivot link hole of both the first arm and the second arm.

3. The tightenable joint of claim 1, wherein the tightening force is a tensile force on the pivot link.

4. The tightenable joint of claim 1, wherein the tightening force is a compressive force on the pivot link.

5. The tightenable joint of claim 1, wherein the clamp body comprises a loosened stop for the handle to prevent over-rotation of the handle in a loosening direction and a tightened stop for the handle to prevent over-rotation of the handle in a tightened direction.

6. The tightenable joint of claim 1, further comprising a tolerancing mechanism to adjust the position of the pivot link relative to the rigid handle to account for a tolerance stack of parts in the clamp.

7. The tightenable joint of claim 1, wherein the handle rolls on the clamp body at the frictional bearing point during tightening of the clamp.

8. The tightenable joint of claim 1, wherein the handle pivot axis translates relative to the clamp body during tightening of the clamp.

9. The tightenable joint of claim 1, wherein the handle pivot axis is fixed relative to the clamp body during tightening of the clamp.

10. A tightenable joint for a surgical retraction system, the tightenable joint comprising:
a clamp body defining a first opening for receiving a clamped rod, the clamp body comprising a first arm and a second arm operatively coupled together to change dimensions of the first opening to receive the clamped rod in a loosened clamp position or in a tightened clamp position;
a pivot link supporting a tightening force between the first arm and the second arm when the clamp is in the tightened clamp position, the pivot link having a handle coupled end, the pivot link defining a pivot link axis;
a handle pivoting relative to the clamp body between the loosened clamp position and the tightened clamp position, the handle being coupled to the handle coupled end of the pivot link;
wherein movement of the handle from the loosened clamp position to the tightened clamp position causes the handle receiving end of the pivot link to move with an axial component distance along the pivot link axis and a lateral component distance transverse to the pivot link axis, wherein the lateral component distance is greater than the axial component distance.

11. A tightenable joint for a surgical retraction system, the tightenable joint comprising:
a clamp body defining a first opening for receiving a clamped rod, the clamp body comprising a first arm and a second arm operatively coupled together to change dimensions of the first opening to receive the clamped rod in a loosened clamp position or in a tightened clamp position;
a pivot link supporting a tightening force between the first arm and the second arm when the clamp is in the tightened clamp position, wherein the pivot link extends generally vertically;
a handle pivoting relative to the clamp body about a handle pivot axis between the loosened clamp position and the tightened clamp position, the rigid handle being coupled to the pivot link at a pivot link coupling point, wherein, during throw of the handle from the loosened clamp position to the tightened clamp position, the pivot link coupling point remains vertically oriented on the same side of the handle pivot axis (point 5 is always above point 1).

12. A tightenable joint for a surgical retraction system, the tightenable joint comprising:
a clamp body; and
a lower jaw having a projecting lip, the lower jaw being coupled relative to the clamp body such that the tightenable joint can be clipped onto a surgical retractor ring which is rectangular in cross-section with the projecting lip wrapping around the surgical retractor ring to snap the tightenable joint onto the retractor ring with the tightenable joint contacting four sides of the surgical retractor ring.

13. The tightenable joint of claim 12, wherein the projecting lip comprises a sloped entry surface, such that the lower jaw can be deflected by contacting the sloped entry surface onto the surgical retractor ring.

14. The tightenable joint of claim 12, wherein the clamp body comprises a sloped entry surface for sliding of the surgical retractor ring, such that the lower jaw can be deflected by sliding the clamp body's sloped entry surface on the surgical retractor ring.

15. The tightenable joint of claim 12, further comprising a spring for snapping the projection lip around the retractor ring after the joint is advanced to a seating position relative to the clamp body.
